(19) United States
(12) Patent Application Publication
(10) Pub. No.: US 2013/0158522 A1
(43) Pub. Date: Jun. 20, 2013

(54) DEVICE FOR PROTECTING AND CLEANING THE BLADDER LUMEN

(71) Applicants: Thomas Lisowsky, Monheim (DE); Heinz-Dieter Held, Huellhorst (DE)

(72) Inventors: Thomas Lisowsky, Monheim (DE); Martin Rahe, Huellhorst (DE)

(73) Assignee: AGXX INTELLECTUAL PROPERTY HOLDING GMBH, Berlin (DE)

(21) Appl. No.: 13/806,315

(22) PCT Filed: Jun. 22, 2011
(86) PCT No.: PCT/EP11/60459

§ 371 Date: Mar. 7, 2013

(30) Foreign Application Priority Data
Jun. 23, 2010 (DE) 10 2010 024 820.7

(51) Int. Cl. A61M 25/00

(52) U.S. Cl. CPC A61M 25/0017
USPC 604/544

(57) ABSTRACT

The invention relates to a device (1), in particular for protecting and for cleaning the bladder lumen, consisting of at least one functional body (2) which can be spatially expanded in a variable manner and can be modified at least partially from a small state into a larger state. Said device (1) protects the bladder lumen from bacterial invasion when the functional body (2) is inserted into the bladder and helps an infected bladder during mechanical cleaning and subsequent healing. The invention also relates to the use of the device (1) for medical, in particular prophylactic and therapeutic purposes.

### BACKGROUND OF THE INVENTION

The invention relates to a device, in particular for protecting and cleaning the urinary bladder lumen, which at least comprises a functional body. The invention further concerns the use of the device for medical, in particular prophylactic and therapeutic, purposes.

### Prior Art

Microbial contaminations of urine and urinary bladder as well as urinary bladder epithelium cause various and severe disorders and costs related thereto. To date, irrespective of the use of strong medicaments, it was not possible to effectively protect the urinary bladder against colonization with germs or to support healing of an already germ-infested urinary bladder.

### DESCRIPTION OF THE INVENTION

It is the object of the invention to render protection of the urinary bladder and its lumen against microbial colonization and the support of healing of infectious diseases possible, irrespective of the use of medicaments.

According to the invention the object is achieved by a device of the initially mentioned kind which is characterized in that the spatial dimensions of the functional body are variable, wherein the functional body is at least partially transformable from a smaller state to a larger state. As functional body inserted into the urinary bladder the device according to the invention protects the bladder against colonization with germs and supports mechanical cleaning of a germ-infested urinary bladder as well as subsequent healing. Due to this approach, the device according to the invention is thereby able to prevent settling in of germs with the bladder's mucosa. It is an advantage of the device according to the invention that the entrance from the urethra and the ureters into the space of the bladder is mechanically protected. Migration of the germs via the urethra into the bladder is hereby prevented and germs that are already present in the bladder are deterred from climbing into the kidneys. This principle of mechanical protection of entrances is ensured by insertion of a sufficiently large contact area into the areas of the urinary bladder that are particularly in need of protection. To this end, the functional body is at least partially transformable from a smaller state, within which it can be inserted into the urinary bladder via the urethra as in case of a catheter, to a larger state so that its spatial dimensions are larger within the urinary bladder and it is able to exhibit its protective and cleaning function. For insertion into the urinary bladder via the urethra, the functional body is reduced to a diameter of approximately 5 mm. A diameter of about 5 mm corresponds to the established size of catheters that are inserted via the urethra. The geometric problem of an enlargement of the functional body from about 5 mm to the maximum size of the functional body in the urinary bladder after insertion and unfolding is solved by the device according to the invention.

In an advantageous embodiment of the invention it is provided that the functional body at least comprises a material that is permeable to liquids, preferably a material having holes or pores. Thus, the functional body is permeable for urine so that the physiological functions of the urinary bladder are not impaired.

In order to facilitate the transformation of the functional body from the smaller state to the larger state and to enable an optimal spatial expansion of the functional body within the urinary bladder, it is provided in an advantageous embodiment of the invention that the material of the functional body is at least partially elastic.

Preferably, the material of the functional body is selected from the group consisting of close-meshed tissue, porous foil, fine-pored foam, and the like. The material of the functional body has to be selected such that it is indeed permeable for liquids but at the same time holds back pathogen germs. Moreover, the material has to be compatible with the human body and may not cause allergies.

In a particularly advantageous embodiment of the invention it is provided that the material of the functional body is a self-inflating material so that the functional body is independently transformed to the larger state in the urinary bladder without external intervention.

In order to avoid an additional intervention for removing the functional body, it is provided in an advantageous embodiment of the invention that the functional body is biologically degradable.

In a further advantageous embodiment of the invention it is provided that the functional body is at least partially moistened and/or coated with an antimicrobial substance. In this embodiment, the germs are killed upon contact with the surface of the functional body so as to avoid an accumulation of germs in the urinary bladder and expedite healing.

In order to design the functional body as compact as possible in the smaller state and to facilitate the transformation to the larger state, it is provided in an advantageous embodiment of the invention that the functional body is divided into at least two segments, preferably 4 to 32 segments. Thereby, the respective inner layers of the functional body are preferably arranged such that they close the space between the segments located in front of them. Since the distances between the segments increase upon increase of the functional body's volume, the segments have to overlap in the smaller, folded state. The proposed principle of configuration including segments and layers considers the geometric problem of an enlargement of the functional body from about 5 mm (at a preferred form of insertion by a guiding pin) to the maximum size of the functional body in the bladder after insertion and unfolding.

Preferably, the functional body is at least partially folded, rolled and/or pressed in the smaller state. The functional body may thereby have a diameter of at most 4-6 mm, preferably about 5 mm, in the smaller state. In the larger state, the functional body is preferably at least partially unfolded, unrolled and/or unwound.

In an advantageous embodiment of the invention it is provided that the functional body includes at least two layers, preferably an inner layer and an outer layer.

Herein, the inner layer may particularly be provided with a, preferably temporary, gas and/or water proof coating. If unfolding of the functional body is externally initiated, for example, by a fluid (liquid, gas) by means of a syringe or other devices, it is ensured by the coating of the inner layer that a pressure sufficient for unfolding can build up within the functional body. Preferably, the coating independently disintegrates after a certain period of time so that gas and liquid exchange within the urinary bladder is not impaired.

The outer layer may have a structured surface and/or preferably be provided with pimples, embossments, or the like so that germs and biofilms colonizing the inner wall of the bladder can be mechanically detached and removed. To this end, the low elastic operating forces of the functional body's material are used to slightly press the structured surface of the outer layer against the bladder wall. Because of the steady change of the bladder's volume during purging and filling a mechanical movement is generated, which causes the detachment and removal of germs and biofilms due to the close contact between the surface of the functional body and the bladder's wall as well as very slight abrasion forces.

In order to facilitate insertion of the device according to the invention into the urinary bladder and in particular guiding through the urethra, it is provided in an advantageous embodiment of the invention that at the outside the functional body is provided with a, preferably forming, cover.

In an advantageous embodiment of the device according to the invention it is further provided that additionally a guiding element, preferably a guiding pin, is provided, which causes sufficient rigidity of the functional body. The functional body may be reduced to a diameter of about 5 mm by folding and winding around a guiding pin, e.g. for insertion into the urinary bladder through the urethra.

The invention further generally relates to the use of the device for medical, in particular prophylactic and therapeutic, purposes. Thereby, the invention particularly comprises the use of the device according to the invention as a type of catheter for protecting and cleaning the urinary bladder lumen. Preferably, the device according to the invention is used in the form of a functional body which has a variable size and, closely folded, allows for insertion into the urinary bladder through the urethra according to known catheters.

The invention is further described in detail by means of the drawings set forth hereunder.

### BRIEF DESCRIPTION OF THE DRAWINGS

FIG. 1: Schematic representations of a preferred embodiment of a device according to the invention—including

FIG. 1A: Overview of the structure of the device;

FIG. 1B: Details of the structure of the device according to FIG. 1A;

FIG. 1C: Functional body folded around supporting pin;

FIG. 1D: Schematic of the segmentation of the functional body;

FIG. 1E: Cross-sectional view of the device according to FIG. 1C;

FIG. 1F. Longitudinal section of the device according to FIG. 1C.

FIG. 2: Schematic representations of the use of a device according to the invention—including

FIG. 2A: Schematic of a completely unfolded functional body in the urinary bladder;

FIG. 2B: Schematic of the insertion of the functional body into the urinary bladder;

FIG. 2C: Details of the completely unfolded functional body in the urinary bladder according to FIG. 2A;

FIG. 2D. An exemplary syringe for unfolding the functional body.

FIG. 3: Schematic representation of the use of a device according to the invention as a supplement of a functional body already unfolded in the urinary bladder by insertion of a second functional body by means of a supporting pin.

### DESCRIPTION OF EXEMPLARY AND PREFERRED EMBODIMENTS

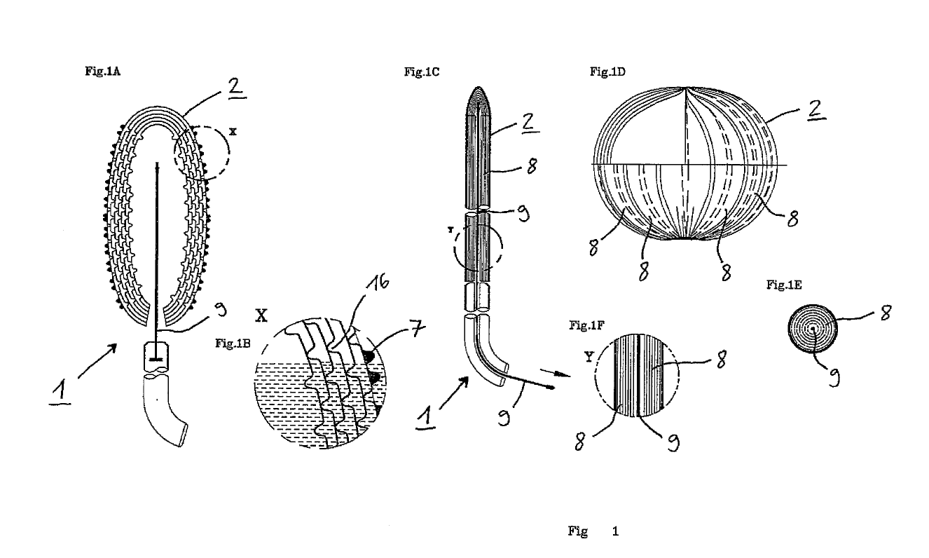

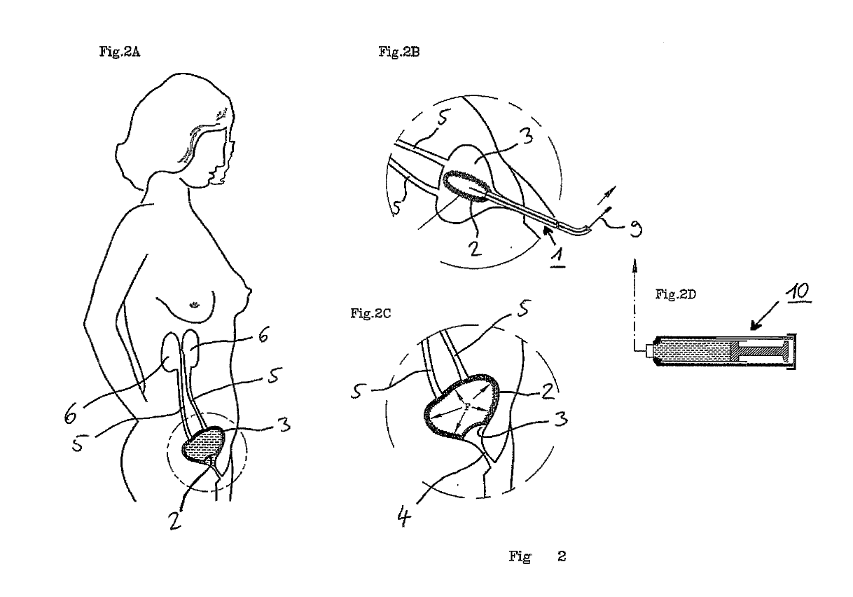

FIG. 1 shows schematic representations of a preferred embodiment of a device according to the invention. The device 1 comprises a functional body 2 which may comprise, for example, slightly elastic unified fine thread, porous foil or open-pored foam, preferably provided with an antimicrobial coating. This functional body 2 may be inserted into the urinary bladder 3 of a human being as temporary implant (FIG. 2A to 2C). A functional body 2, which shall completely fill the whole bladder space so as to be fully functional, could not pass the urethra 4 as a catheter due to its large volume. Consequently, insertion is preferably employed in a folded, rolled state as in case of a catheter (FIG. 1C). After insertion into the urinary bladder 3 the functional body 2 is unfolded (FIG. 1A and 1D) so that, in close contact to the wall of the urinary bladder and by filling the bladder lumen, a mechanical cleaning may occur utilizing the bladder's in/exhalation (FIG. 2A and 2C). The continuous contractions of the bladder lumen together with the elasticity of the functional body 2 and its filling of the entire bladder lumen lead to a mechanical cleaning of the bladder's wall which is prone to colonization with germs. The close-meshed, fine-pored tissue of the functional body 2 enables free passage of the urine (FIG. 1B). At the same time, the functional body 2 blocks and prevents the climbing migration of germs from the urethra 4 into the urinary bladder 3 and from the urinary bladder 3 into the ureters 5 and kidneys 6 (FIGS. 2A and 2C). In an advantageous embodiment of the invention, an antimicrobial coating of the tissue of the functional body may ensure that the surface of the functional body 2 is kept sterile and germs cannot grow at the surface of the functional body 2. Germs which get in contact with the surface of the functional body 2 are killed then. The structure of the tissue of the functional body 2 enables the formation of a large contact area having a respectively large area with antimicrobial coating keeping the tissue region of functional body 2 sterile. The close-meshed, fine-pored tissue of functional body 2 allows for free passage of the urine. Due to the close mesh of the tissue in connection with a plurality of miniaturized cells, there is only a very small distance between the urinary bladder epithelia colonized with germs and germs in the urine.

The outer layer of the functional body 2 is preferably provided with tiny, relatively inherently stable pimples 7 (FIG. 1B) which mechanically detach and remove germs and biofilms colonizing the inner wall of the bladder. To this end, the low elastic operating forces of the tissue material are used to slightly press the structured surface (pimples 7) against the bladder wall. Because of the steady change of the bladder's volume during purging and filling a mechanical movement is generated, which causes the detachment and removal of germs and biofilms due to the close contact between the structured surface of the functional body 2 and the bladder's wall as well as very slight abrasion forces.

In an advantageous embodiment of the invention, the functional body 2 of the device 1 according to the invention is divided into multiple segments 8 which enable a compact configuration of the functional body 2 in the smaller state and facilitate the transition to the larger state (FIG. 1C to 1F). The respective inner layers of the functional body 2 are thereby preferably arranged such that they close the space between the segments 8 located in front of them. Since the distances between the segments 8 increase upon increase of the functional body's volume, the segments 8 have to overlap in the smaller, folded state. Due to this specific configuration of the segments 8 it is achieved that as much as possible of the entire surface of the bladder is covered without gaps of effect. To this end, low operating forces based on a predetermined elasticity of the tissue material have to ensure that the surface of the functional body 2 may contact the inner wall of the bladder almost completely. Access of the germs to the bladder mucosae and particularly to ureters 5 and urethra 4 may be avoided in any case by an antimicrobial coating. In the inner space of the functional body 2, germs that may enter bladder space almost exclusively via the urethra 4 do not have pathogen relevance.

The functional body 2 comprises a material that is preferably provided with bulges 18 being embossed into the tissue. The form stability of the material should be determined such that the structures almost disappear upon pressing into the smaller state (catheter shape).

The device 1 according to the invention may further comprise a guiding element stabilizing the functional body 2. In order to provide the functional body 2 with sufficient rigidity for inserting through the urethra 4, it is beneficial to wrap the functional body 2 within the relevant region, for example, around a thin guiding pin 9, e.g. made of metal or plastics (FIG. 1C, 1D and 1E). After insertion into the urinary bladder 3 the guiding pin 9 may be drawn out again (FIG. 2B) so as to initiate unfolding of the functional body 2 through the resulting canal by inserting a fluid using a syringe 10 (FIG. 2D) or other devices. With this approach it is necessary that the inner functional layer is made water and/or gas proof during installation by means of a temporary coating (e.g. PVA, acrylate or other self-degrading materials).

The functional body 2 should only stay in the urinary bladder 3 for a limited period of time of about at most 3 months so as to initiate and ensure cleaning and healing process. After cleaning the urinary bladder 3 and the bladder lumen of germs, the functional body 2 is preferably removed. The functional body 2 may be implemented as a kind of biologically degradable material so that it does not have to be removed by means of another external intervention. Thus, the material gradually disintegrates in the urine into tiny particles which are flushed out with the urine via the urethra 4. The degradation of the supporting material that is merely covered by an antimicrobial coating can be established much easier as if compound materials were used, because the antimicrobial coating is not mingled with the supporting polymer and hence cannot exhibit an interfering effect.

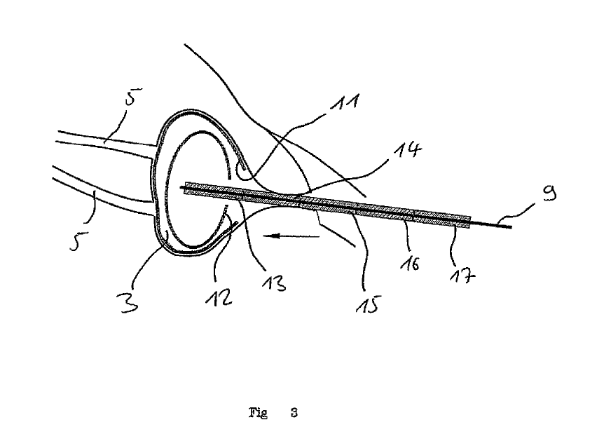

FIG. 3 shows a further exemplary embodiment of the device according to the invention as well as its use. Consecutively, single elements 11, 12, 13, 14, 15, 16, 17 of the functional body 2 are respectively pushed over the guiding pin 9 into the inside of the preceding functional body element. By dividing and folding elements 11, 12, 13, 14, 15, 16, 17 the catheter may be kept very thin. Thus, if a larger contact area is needed, the second, smaller element 12, which is folded around the guiding pin 9, can be inserted from below via the urethra 4 into the cavity of the larger element 11. Utilizing the memory effect of preformed materials or by means of dissolving reinforcing substances or the like, the whole structure of the functional body assembles in the bladder space and flexibly adapts to the urinary bladder epithelia of the bladder's periphery, for example, by means of mechanical pretension. According to this approach, differently functionalized layers may be established as well. For instance, the outer membrane may thus be a blister foil being provided with an additional antimicrobial principle for sterilizing the urinary bladder 3 and the inner surface of the urinary bladder, respectively.

The constructive implementation of the device 1 by means of the close meshed tissue of the functional body 2 with a multilayered structure and minimum distances, preferably including a microbial coating, may be technically established using known materials and methods.

### REFERENCE NUMBERS

1 Device

2 Functional body

3 Urinary bladder

4 Urethra

5 Ureter

6 Kidney

7 Pimple

8 Segment

9 Guiding pin

10 Syringe

11 Element

12 Element

13 Element

14 Element

15 Element

16 Element

17 Element

18 Bulge

1. Device for protecting and cleaning an urinary bladder lumen, at least comprising:
a functional body whose spatial dimensions are variable, wherein the functional body is at least partially transformable from a smaller state to a larger state.

2. The device according to claim 1, wherein the functional body at least partially comprises a material that is permeable to liquids.

3. The device according to claim 1, wherein the material of the functional body is selected from the group consisting of close-meshed tissue, porous foil, and fine-pored foam.

4. The device according to claim 1, wherein the material of the functional body is a self-inflating material.

5. The device according to claim 1, wherein the functional body is biologically degradable.

6. The device according to claim 1, wherein the functional body is at least partially moistened and/or coated with an antimicrobial substance.

7. The device according to claim 1, wherein the functional body is divided into at least two segments.

8. The device according to claim 1, wherein in the smaller state the functional body is at least partially folded, rolled and/or pressed.

9. The device according to claim 1, wherein in the smaller state the functional body has a diameter of at most 4-6 mm.

10. The device according to claim 1, wherein the functional body includes at least two layers.

11. The device according to claim 1, wherein, at the outside, the functional body is provided with a cover.

12. The device according to claim 1, further comprising a guiding element, is provided.

13. as Method for protecting and cleaning an urinary bladder lumen comprising
providing the device of claim 1, and
protecting and cleaning the urinary bladder lumen, wherein the functional body of the device is at least partially transformed from a smaller state to a larger state during said protecting and cleaning.

14. The device according to claim 2, wherein the material permeable to liquids is a material having holes or pores.

15. The device according to claim 7, wherein the functional body is divided into at least 4 to 32 segments.

16. The device according to claim 9, wherein the diameter is the smaller state is about 5 mm.

17. The device according to claim 10, wherein the at least two layers comprise an outer and an inner layer and preferably an inner layer and an outer layer, and the inner layer is provided with a gas and/or water proof coating, and/or the outer layer has a structured surface.

18. The device according to claim 17, wherein said gas and/or water proof coating is temporary and/or the structured surface is provided with pimples or embossments.

19. The device according to claim 11, wherein the cover is a forming cover.

20. The device according to claim 12, wherein the guiding element is a guiding pin.
